(19) United States
(12) Patent Application Publication
(10) Pub. No.: US 2014/0081087 A1
(43) Pub. Date: Mar. 20, 2014

(54) MOBILE DEVICE SYSTEM ACTIVELY CAPTURING PHYSIOLOGICAL PARAMETERS

(71) Applicant: Shan-Yi YU, New Taipei City (TW)

(72) Inventor: Shan-Yi YU, New Taipei City (TW)

(21) Appl. No.: 13/620,183

(22) Filed: Sep. 14, 2012

(51) Int. Cl. A61B 5/0205; A61B 5/0402; A61B 5/145; A61B 5/00; A61B 5/01

(52) U.S. Cl. 600/301; 600/300

(57) ABSTRACT

The present invention relates to a mobile device system actively capturing physiological parameters which comprises a suite of master software and a physiological measurement module: the master software actively sends master control signals through the mobile device's first wireless communications module and receives as well as present wireless physiological signals so that the physiological measurement module which is controlled by the mobile device measures physiological data and deliver them to the mobile device for display. As such, the mobile device can be used to actively control the physiological measurement module, capturing and saving physiological data, and actively presenting physiological data on the mobile device's display.

### BACKGROUND OF THE INVENTION

1. Field of the Invention

The present invention relates to a mobile device system actively capturing physiological parameters, particularly a system which depends on a portable mobile device to actively control and capture physiological parameters.

2. Description of the Related Art

It has seemingly become common sense of modern people to regularly measure physiological data because chronic diseases induced by changes in eating habits or life patterns such as premonitory hyperglycemia, hypertension and hyperlipidemia particularly have seriously affected health of people in a modern society. Principally, existing physiological measurement instruments can be divided into three parts, that is, physiological measurement unit 1, microcontroller module 2 and input/output module 3: the physiological measurement unit 1 depends on human body measurement to capture a real-time physiological signal; the microcontroller module 2 is used to control the physiological measurement unit 1 and the input/output module 3; the input/output module 3 is provided with a display as a surveyor's operation interface to present data and as a connection port to input or store data. With a real-time physiological signal captured in human body measurement by the physiological measurement unit 1, the signal which is processed in the microcontroller module 2 becomes real-time physiological data and is presented on a display of the input/output module 3 for access, storage or printing. The cost to design and manufacture a physiological measurement instrument is high in virtue of more and more personalized and customized physiological measurement tools in which the costly large-size input/output module display's price is drastically fluctuating and not effectively controlled by one manufacturer. In addition, a manufacturer still makes efforts to design and develop operation interfaces meeting various demands of surveyors who depend on different healthy states to take notice of distinct physiologic parameters. However, an operation interface which meets specific demands but is too complicated has insidiously perplexed a surveyor who is unfamiliar with the interface.

With powerful mobile devices popularized and platforms for software download matured, the solution to avoid a costly physiological measurement instrument and supply a customized or even personalized operation interface by means of effective use of a common mobile device for easily capturing and carrying measured physiological data deserves to be studied by persons skilled in the art.

### SUMMARY OF THE INVENTION

The present invention is intended to provide a mobile device system actively capturing physiological parameters wherein the system is based on a mobile device, which is capable of actively controlling a physiological measurement module to capture and save physiological data and present them on the mobile device's display, and comprises: A suite of master software which is installed in the mobile device with a display in advance as well as at least a first wireless communications module and is capable of actively sending master control signals and receiving as well as presenting wireless physiological signals via the mobile device's first wireless communications module; a physiological measurement module which is not equipped with an internal display, captures at least one type of physiological data by measuring a human body, and is provided with at least a second wireless communications module comprising at least a physiological signal transmission unit in order to send physiological data as a wireless signal that can be received by the first wireless communications module and delivered to the master software for saving data and actively presenting them on the mobile device's display. A control signal receiver unit which actively receives control signals from the mobile device in order to trigger the physiological measurement module for human body measurement. Based on the above principal features, the mobile device can be a smart mobile phone, a tablet computer, a notebook computer or a simple computer. Based on the above principal features, the first wireless communications module and the second wireless communications module can be a wireless communications module comprising BLUETOOTH, IR, WIFI (Wireless Fidelity), Radio Frequency IDentification (RFID for short), or Near Field Communication (NFC for short). Based on the above principal features, the master software allows personal information to be input. Based on the above principal features, the physiological data comprises body temperature, pulse rate, blood pressure, blood glucose, blood oxygen saturation, electrocardiogram (EKG or ECG), and respiration parameter.

### BRIEF DESCRIPTION OF THE DRAWINGS

FIG. 1 is a schematic view for architecture of a physiological measurement instrument based on prior arts.

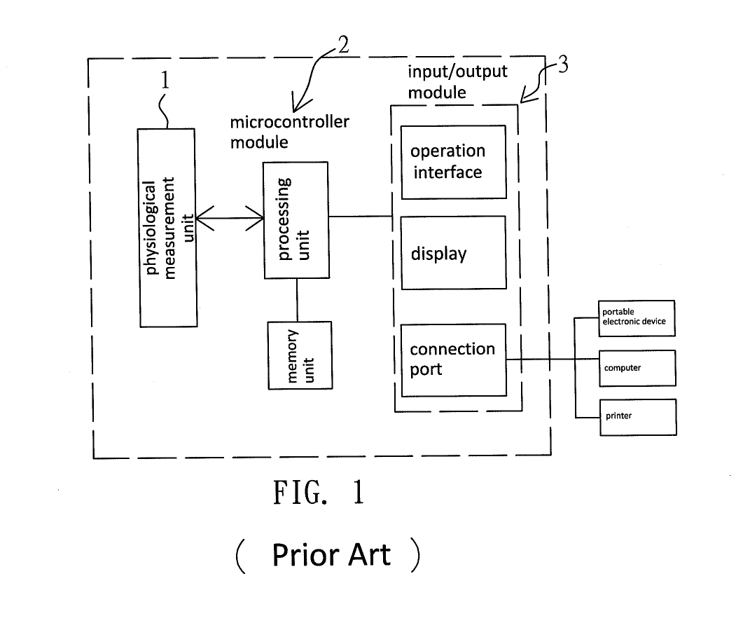

FIG. 2 is a schematic view for architecture of the present invention of a mobile device system actively capturing physiological parameters.

### DETAILED DESCRIPTION OF THE PREFERRED EMBODIMENTS

To achieve the above purposes, the preferred embodiments and appended drawings are presented hereinafter to explain technical measures and effects of the present invention.

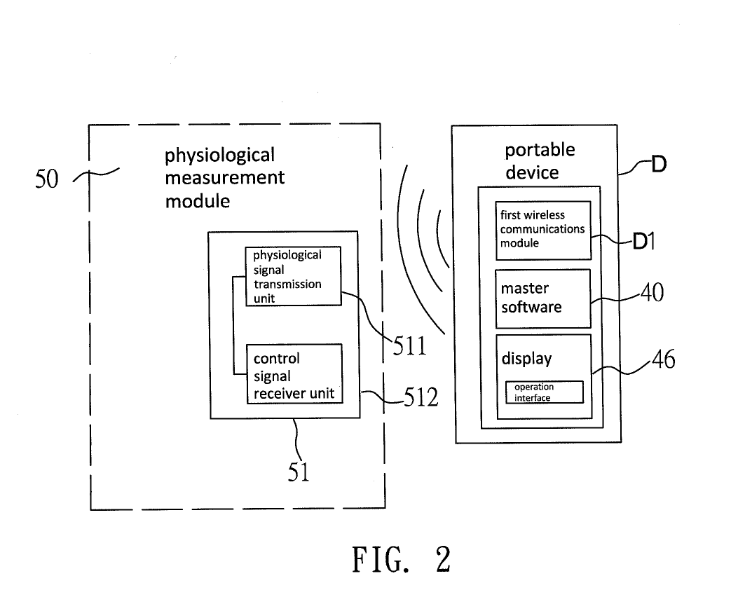

A mobile device D in the present disclosure is not limited to one specific device but one information technology device (an IT device in which there are CPU and memories installed) with a portable wireless communications module and a display such as smart mobile phone, tablet computer, notebook computer or simple computer (e.g., EeePC). A schematic view for architecture of the present invention of a mobile device system actively capturing physiological parameters is shown in FIG. 2. As shown in FIG. 2, the present invention provides a mobile device system actively capturing physiological parameters which comprises a suite of master software 40 and a physiological measurement module 50: the physiological measurement module 50 does not comprise an internal display but a second wireless communications module 51; the mobile device D is provided with at least a first wireless communications module D1; the master software 40 actively employs the first wireless communications module D1 in the mobile device D to control (a) the physiological measurement module 50 for human body measurement and (b) the second wireless communications module 51 to deliver data to the mobile device D for data storage, display and analyses which are further explained hereinafter: The master software 40 which is installed in the mobile device D in advance is able to actively define the wireless communications module in the mobile device D as the first wireless communications module D1 which is used to actively send master control signals and receive wireless physiological signals from the physiological measurement module 50.

With the master software 40 actually activated (executed), the first wireless communications module D1 is able to actively search and match the second wireless communications module 51 nearby and control the physiological measurement module 50 by means of actively transmitted master control signals. The physiological measurement module 50 is able to measure a human body to capture at least one type of physiological data such as body temperature, pulse rate, blood pressure, blood glucose, blood oxygen saturation, electrocardiogram (EKG or ECG), and respiration parameter, that is, the physiological measurement module comprises functions of the following gauges such as thermometer, pulsimeter, sphygmomanometer, blood glucose meter, oximeter, heart rate monitor, and respiration parameter meter without a display; furthermore, the physiological measurement module 50 is provided with at least the second wireless communications module 51 in which there are at least a physiological signal transmission unit 511 and a control signal receiver unit 512 installed: the physiological signal transmission unit 511 is able to send physiological data as a wireless signal which is received by the first wireless communications module D1 of the mobile device, delivered to the master software 40 for saving data and presenting them on a display of the mobile device D; the control signal receiver unit 512 actively receives control signals from the mobile device D in order to trigger the physiological measurement module 50 for human body measurement. Moreover, the first wireless communications module D1 and the second wireless communications module 51 can be a wireless communications module comprising BLUETOOTH, IR, WIFI (Wireless Fidelity), Radio Frequency Identification (RFID for short), or Near Field Communication (NFC for short). The present invention of a mobile device system is executed in a first stage and a second stage. First stage: The master software 40 which has been activated (executed) is able to actively trigger a matching function of the first wireless communications module D1 for searching and matching the second wireless communications module 51 nearby. However, the method for search and matching is beyond the present disclosure and not presented herein. With matching completed, the first wireless communications module D1 is capable of actively sending control signals to control the physiological measurement module 50 (so-called initialization) so that a surveyor operates the master software 40 in the mobile device D to send master control signals through the first wireless communications module D1 and trigger the physiological measurement module 50 for measurement.

Second stage: With real-time physiological data successfully received by the physiological measurement module 50, the physiological data as a wireless signal is sent by the physiological signal transmission unit 511 in the second wireless communications module 51, received by the first wireless communications module D1 in the mobile device D, and delivered to the master software 40 which is able to complete data storage and present them on a display 46 of the mobile device D.

In an appropriate embodiment, the above physiological signal transmission unit 511 is capable of encrypting and sending the physiological data captured by the physiological measurement module 50 as a wireless signal which can be received and decrypted as the physiological data by the first wireless communications module D1 in the mobile device D and delivered to the master software 40 which completes data storage and presents them on a display 46 of the mobile device D. In an appropriate embodiment, the mobile device D comprises a speaker for speaking out physiological data which can be easily heard by users. In an appropriate embodiment, the mobile device D allows information to be input, and delivers the input information to the physiological measurement module 50 through the first wireless communications module D1 for data storage.

In an appropriate embodiment, the physiological measurement module 50 allows information to be input, and delivers the input information to the mobile device D through the second wireless communications module 51 for data storage.

In an appropriate embodiment, the master software 40 is able to automatically identify physiological data from various measurement components in order to execute corresponding controls.

In an appropriate embodiment, the master software 40 is able to automatically identify physiological data from various measurement components and link more than one physiological measurement module 50 in order to execute corresponding controls via wireless connections.

In an appropriate embodiment, the master software 40 is able to control operations in the physiological measurement module 50, for instance, various steps for measuring blood pressure can be shown with icons, pictures, texts or sound through the master software 40 and easily operated by one user via the mobile device D.

Furthermore, the master software 40 which is effective in actively sending control signals and receiving and presenting wireless physiological signals in the present invention still has other functions such as saving measured history data, allowing an individual's information (e.g., height, age, gender or expected BMI (Body Mass Index)) to be entered, and listing, analyzing and calculating physiological history data in order to generate a statistical chart or graph for a surveyor's references to control states. For instance, blood pressure as one physiological parameter measured day and night weekly can be expressed in a trend chart which is clearly checked by one user.

In an appropriate embodiment, the master software 40 is able to manage an individual's at least one physiological parameter.

In an appropriate embodiment, the master software 40 further comprises medical information related to the physiological parameters such as scope of normal blood pressure specified by WHO (World Health Organization) and notes for measurement of blood pressure.

It can be seen from above descriptions that the present invention has two advantages as follows:

1. The present invention is characteristic of physiological data controlled and displayed by a surveyor's mobile device in both the first stage and the second stage, that is, the physiological measurement module 50 in the present invention needs neither the extra display 46 nor an operation interface, so the present invention could effectively economizes manufacture costs.

2. The master software 40 in the present invention can be designed to display various physiological data for professional analyses/judgment or one type of specific physiological data on one single operation interface for easy use in accordance with demands of surveyors including physicians, medical staff, general people and senior citizens. The above descriptions present preferred embodiments only which do not limit the scope of the present invention; any equivalent change or improvement based on technical ideas disclosed in the present invention still does not depart from the present invention.

What is claimed is:

1. A mobile device system actively capturing physiological parameters, comprising:
(a) A suite of master software which is installed in said mobile device with a display in advance, said mobile device comprises at least a first wireless communications module, and said master software actively sends master control signals through said mobile device's first wireless communications module and receives wireless physiological signals; (b) a physiological measurement module without an internal display which measures a human body to capture at least one type of physiological data and is provided with at least a second wireless communications module which comprises at least a physiological signal transmission unit sending said physiological data as a wireless signal that can be received by said mobile device's first wireless communications module and delivered to said master software for storing data and actively presenting them on said mobile device's display; (c) a control signal receiver unit which actively receives control signals from said mobile device in order to trigger said physiological measurement module for human body measurement.

2. The mobile device system actively capturing physiological parameters according to claim 1 wherein said physiological data comprises body temperature, pulse rate, blood pressure, blood glucose, blood oxygen saturation, electrocardiogram (EKG or ECG), and respiration parameter.

3. The mobile device system actively capturing physiological parameters according to claim 1 wherein said mobile device can be a smart mobile phone, a tablet computer, a notebook computer or a simple computer.

4. The mobile device system actively capturing physiological parameters according to claim 1 wherein said physiological signal transmission unit encrypts and sends physiological data received by said physiological measurement module as a wireless signal which can be received and decrypted as said physiological data by said mobile device's first wireless communications module, delivered to said master software for storing data and actively presenting them on said mobile device's display.

5. The mobile device system actively capturing physiological parameters according to claim 1 wherein said mobile device comprises a speaker for speaking out physiological data.

6. The mobile device system actively capturing physiological parameters according to claim 1 wherein said first wireless communications module and said second wireless communications module can be a wireless communications module comprising BLUETOOTH, IR, WIFI (Wireless Fidelity), Radio Frequency IDentification (RFID for short), or Near Field Communication (NFC for short).

7. The mobile device system actively capturing physiological parameters according to claim 1 wherein said mobile device allows data to be input and delivered to and saved in said physiological measurement module from said first wireless communications module.

8. The mobile device system actively capturing physiological parameters according to claim 1 wherein said physiological measurement module allows data to be input and delivered to and saved in a mobile device from said second wireless communications module.

9. The mobile device system actively capturing physiological parameters according to claim 1 wherein said master software is able to automatically identify physiological data from various measurement components for corresponding controls.

10. The mobile device system actively capturing physiological parameters according to claim 1 wherein said master software is able to automatically identify physiological data from various measurement components and link more than one physiological measurement module for corresponding controls via wireless connections.

11. The mobile device system actively capturing physiological parameters according to claim 1 wherein said master software is able to control operations of said physiological measurement module.

12. The mobile device system actively capturing physiological parameters according to claim 1 wherein said master software allows an individual's information to be input.

13. The mobile device system actively capturing physiological parameters according to claim 1 wherein said master software is able to list physiological history records.

14. The mobile device system actively capturing physiological parameters according to claim 1 wherein said master software is able to list a statistical chart or graph based on physiological history records.

15. The mobile device system actively capturing physiological parameters according to claim 1 wherein said master software is able to manage an individual's at least one physiological parameter.

16. The mobile device system actively capturing physiological parameters according to claim 1 wherein said master software further comprises medical information related to said physiological parameters.
